(19) United States
(12) Patent Application Publication
(10) Pub. No.: US 2009/0069735 A1
(43) Pub. Date: Mar. 12, 2009

(54) ANKLE GUARD AND BRACE SEGMENT FOR THE ANKLE GUARD

(76) Inventor: Pang-Ching CHIANG, Taipei (TW)

Correspondence Address: PATENTTM.US, P. O. BOX 82788, PORTLAND, OR, 97282-0788, US

(21) Appl. No.: 11/853,813

(22) Filed: Sep. 11, 2007

(51) Int. Cl. A61F 5/01

(52) U.S. Cl. 602/27

(57) ABSTRACT

A brace segment for an ankle guard is worn above and over an ankle to protect the ankle and provide a firm base for a resilient sleeve having two halves. Two brace segments are mounted on the halves of the resilient sleeve and each has a proximal end, a pressing segment and a support cavity. The pressing segment is formed from the proximal end of the brace segment and extends over the ankle. The support cavity is formed by bending the pressing segment to prevent inversion and eversion of the ankle and protect the ankle from knocks. Because the ankle guard has two identical brace segments the ankle guard can be implemented with both feet has lower cost and complexity of manufacture and greater commercial exploitation.

### BACKGROUND OF THE INVENTION

1. Field of the Invention

The present invention relates to an ankle guard, and more particularly to the ankle guard with brace segments worn around and pressing against an ankle to prevent ankle injuries.

2. Description of the Prior Arts

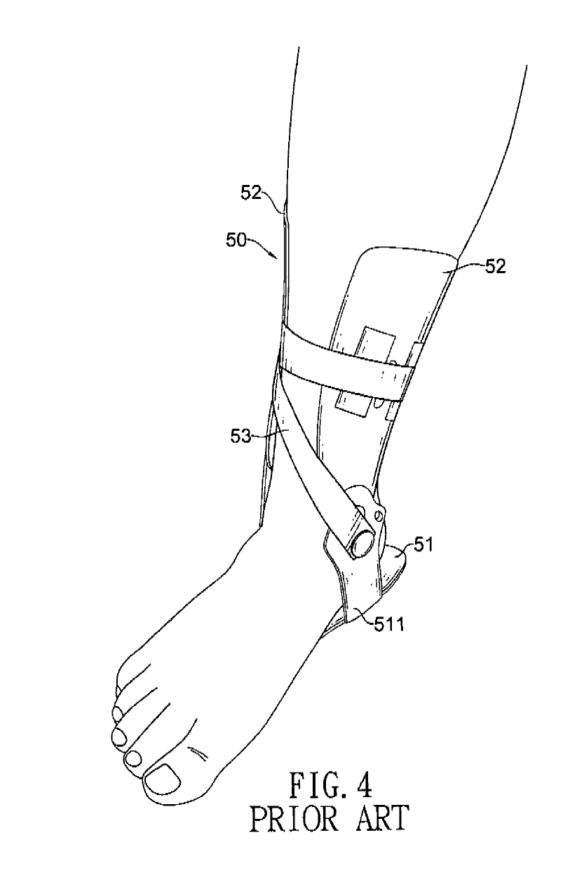

With reference to FIG. 4, a conventional ankle guard (50) is worn around and protects an ankle and has a base segment (51), two brace segments (52) and a mounting belt (53).

The base segment (51) corresponds to a heel and has two pressing braces (511). The pressing braces (511) are formed oppositely on and protrude perpendicularly from the base segment (51) to clamp the ankle from both sides when the ankle guard (50) is worn and each pressing brace (511) has a distal end and an outer surface.

The brace segments (52) are mounted respectively on the distal ends of the pressing braces (511), are worn securely around a lower leg and each brace segment (52) has an outer surface.

The mounting belt (53) is wound tightly around the outer surfaces of the pressing braces (511) and the brace segments (52) to hold and press the pressing braces (511) and the brace segments (52) against the ankle.

Inversion and eversion of the ankle is prevented, however the ankle is not protected well and the base segment (51) being hard is awkward to use with sports shoes. Furthermore the heel of the ankle may slide on the base segment (51) and the strapping does not enclose and protect the ankle, making the ankle susceptible to sprains or injuries during exercise.

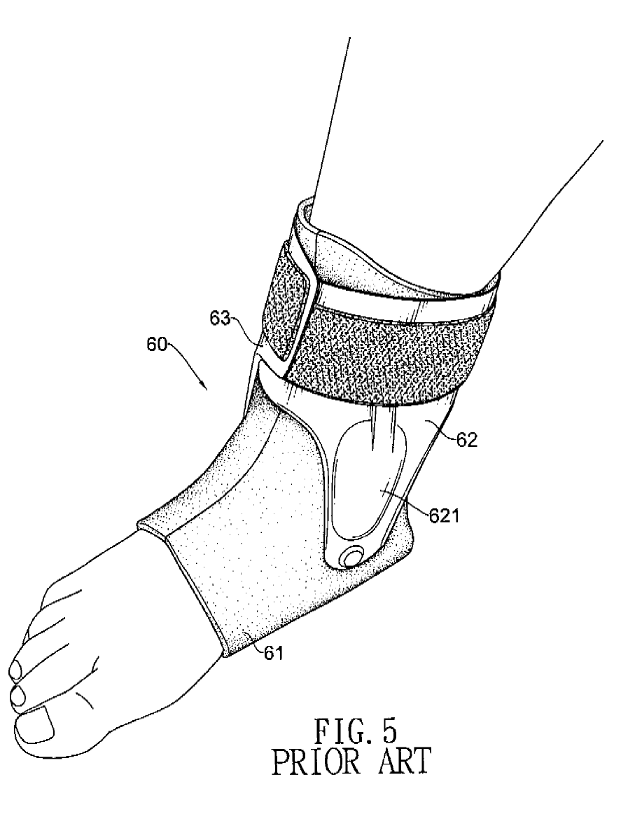

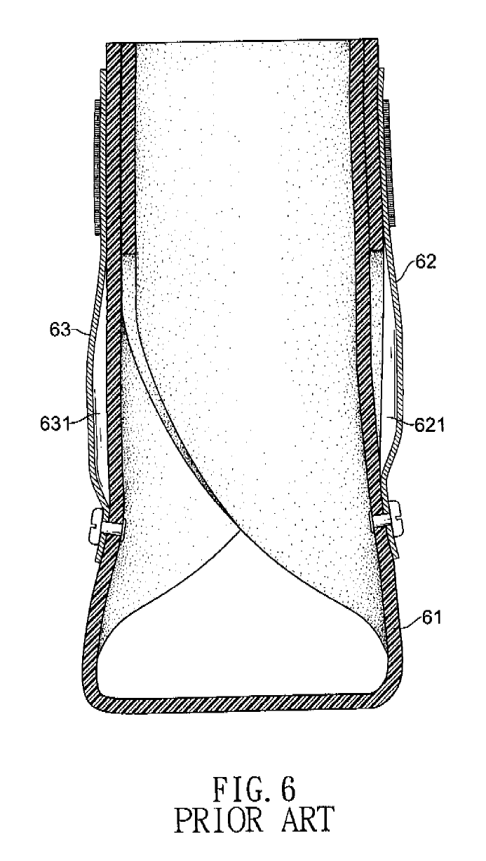

With further reference to FIGS. 5 and 6, another conventional ankle guard (60) is designed to provide a better protection for the ankle and has a resilient sleeve (61), an inner brace segment (62) and an outer brace segment (63).

The resilient sleeve (61) is worn around a foot and the ankle and has an inner half and an outer half.

The inner and outer halves of the resilient sleeve (61) respectively correspond to an inner half and an outer half of the foot and the ankle.

The inner brace segment (62) is mounted pivotally on the inner half of the resilient sleeve (61), is T-shaped, is mounted against and is bent around the inner half of the ankle and has a support (621). The outer brace segment (63) is mounted pivotally on the outer half of the resilient sleeve (61), is T-shaped, is mounted against and is bent around the outer half of the ankle and has a support (631).

The supports (621, 631) are rigid, are formed respectively in the inner and outer brace segments (62, 63), respectively correspond to the corresponding half of the ankle to protect the ankle from inversion and eversion.

This ankle guard (60) provides better protection to the ankle. However, because the ankle guard (60) has two different brace segments (62, 63), cost and complexity of manufacturing the ankle guard (60) are raised. Moreover, because the ankle guard (60) is not symmetrical and one ankle guard (60) only corresponds to either right foot or left foot, the ankle guard (60) can only be used on a corresponding foot. Therefore, the users will have to buy and wear the correct, corresponding ankle guard causing inconvenience to users.

To overcome the shortcomings, the present invention provides an ankle guard to mitigate or obviate the aforementioned problems.

### SUMMARY OF THE INVENTION

The primary objective of the present invention is to provide a brace segment of an ankle guard that is easily manufactured, commercially exploited and worn.

A brace segment for an ankle guard is worn above and over an ankle to protect the ankle and provide a firm base for a resilient sleeve comprising two halves. Two brace segments are mounted on the halves of the resilient sleeve and each has a proximal end, a pressing segment and a support cavity. The pressing segment is formed from the proximal end of the brace segment and extends over the ankle. The support cavity is formed inwardly by shaping the middle of the pressing segment to prevent inversion and eversion of the ankle and protect the ankle from knocks. Because the ankle guard comprises two identical brace segments the ankle guard can be implemented with both feet has lower cost and complexity of manufacture and greater commercial exploitation.

Other objectives, advantages and novel features of the invention will become more apparent from the following detailed description when taken in conjunction with the accompanying drawings.

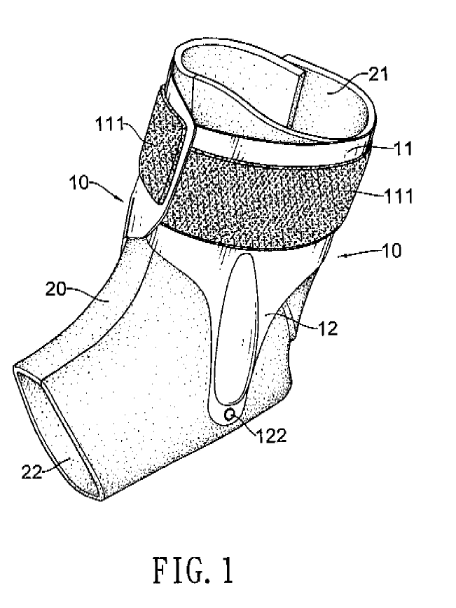

FIG. 1 is a perspective view of an ankle guard in accordance with the present invention;

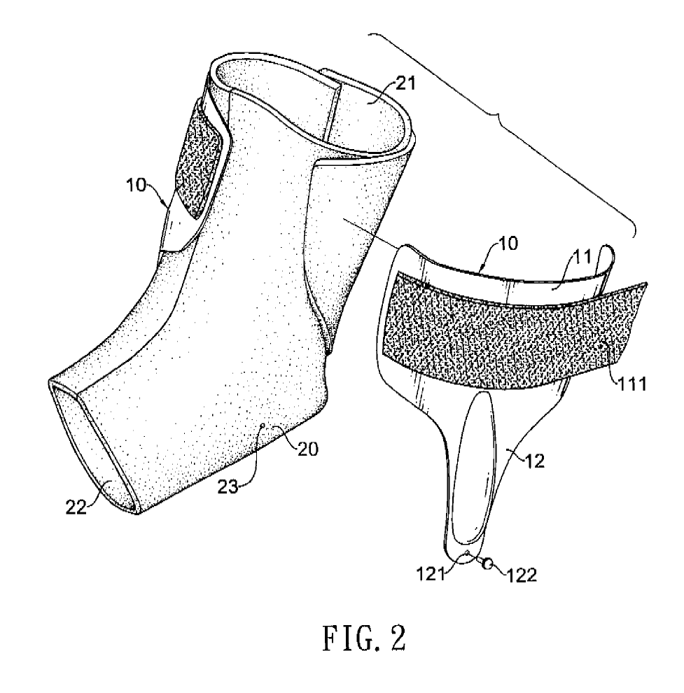

FIG. 2 is a partially exploded perspective view of the ankle guard in FIG. 1;

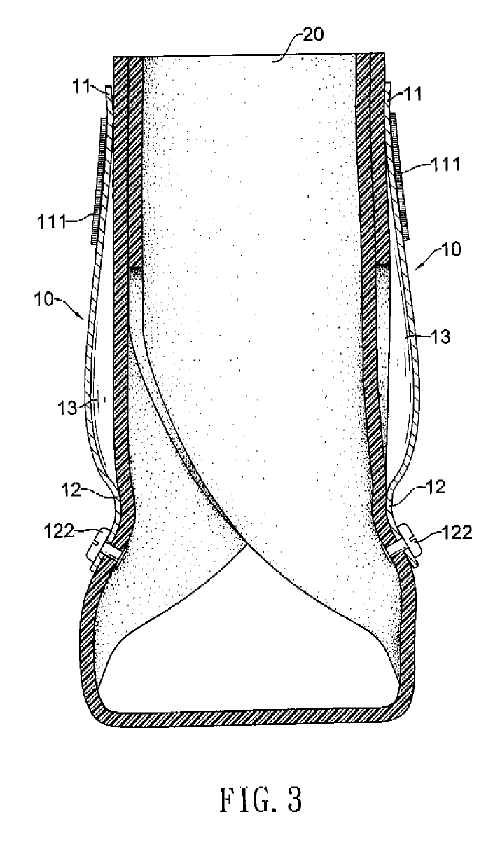

FIG. 3 is a front view in partial section of the ankle guard in FIG. 1;

FIG. 4 is an operational perspective view of a conventional ankle guard in accordance with the prior art;

FIG. 5 is an operational perspective view of another conventional ankle guard in accordance with the prior at; and

FIG. 6 is a front view in partial section of the ankle guard in FIG. 5.

### DETAILED DESCRIPTION OF THE PREFERRED EMBODIMENTS

With reference to FIG. 1, an ankle guard in accordance with the present invention is worn around a foot and an ankle to protect the ankle from injuries, especially injuries caused by inversion and eversion of the ankle and comprises a resilient sleeve (20) and two brace segments (10).

With further reference to FIG. 2, the resilient sleeve (20) is a toeless sock, corresponds to and is worn over the foot and ankle, has a bottom surface, a first end, a second end and two opposite halves and may have two openings (21, 22) and two mounting holes (23).

The bottom surface of the resilient sleeve (20) corresponds to the sole of the foot.

The openings (21,22) are formed respectively in the first and second ends of the resilient sleeve (20) and communicate with each other to allow a user to insert his foot into the ankle guard.

The mounting holes (23) are formed respectively through the halves of the resilient sleeve (20) near the bottom surface of the resilient sleeve (20) and align with each other.

With further reference to FIG. 3, each brace segment (10) is T-shaped, are oppositely mounted pivotally on the halves of the resilient sleeve (20) and has a distal end, a proximal end, a pressing segment (12), a support cavity (13), an optional mounting hole (12 1), an optional fastener (122) and a connecting segment (11).

The pressing segment (12) is formed from the proximal end of the brace segment (10), is mounted pivotally on the resilient sleeve (20), corresponds to and extends over the ankle and has a middle.

The support cavity (13) is form inwardly by shaping the middle of the pressing segment (12) convex, is corresponds to the ankle and prevents inversion and eversion of the ankle, whilst preventing knocks to the ankle.

The mounting hole (121) of the brace segment (10) is formed through the pressing segment (12) near the proximal end of the brace segment (10) and aligns to the mounting holes (23) of the resilient sleeve (20).

The fastener (122) extends through the mounting holes (121, 23) of the brace segment (10) and the resilient sleeve (20) to holds the brace segment (10) pivotally on the resilient sleeve (20) and may be a rivet, bolt, nut or the like and made of non-corrosive metal or plastic.

The connecting segment (11) is formed from the distal end of the brace segment (10), is perpendicular to the pressing segment (12), is mounted around the resilient sleeve (20) and comprises an outer surface and a mounting strip (111). The mounting strip (111) may be a hook and loop fastener, an adhesive or the like and is attached securely to the outer surface of the connecting segment (11) to detachably attach the connecting segments (11) of the brace segments (10) to each other. Therefore, the brace segments (10) adjustably enclose, hold and press the ankle when the ankle guard is worn.

Consequently, the ankle guard provides a good protection for the ankle Moreover, because the ankle guard is made with two identical brace segments (10), cost and complexity of manufacture are reduced, and the ankle guard may be further implemented on either a left or a right foot so is used flexibly by the user and shops need only sell one model, significantly raising commercial exploitation of the ankle guard.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A brace segment for an ankle guard being T-shaped and comprising
a distal end;
a proximal end;
a pressing segment being formed from the proximal end of the brace segment and having a middle;
a support cavity being formed inwardly by shaping the middle of the pressing segment convex; and
a connecting segment being formed from the distal end of the brace segment, being perpendicular to the pressing segment and having an outer surface.

2. The brace segment as claimed in claim 1, wherein the brace segment further comprises
a mounting hole being formed through the pressing segment near the proximal end of the brace segment; and
a fastener extending through the mounting holes of the brace segment.

3. The brace segment as claimed in claim 2, wherein the fastener is a rivet.

4. The brace segment for an ankle guard as claimed in claim 3, wherein the fastener is plastic.

5. The brace segment as claimed in claim 4, wherein the connecting segment further has a mounting strip being attached securely to the outer surface of the connecting segment.

6. An ankle guard comprising
a resilient sleeve being a toeless sock and having
a bottom surface;
a first end;
a second end; and
two opposite halves; and
two brace segments being T-shaped, being oppositely mounted pivotally on the halves of the resilient sleeve and each half having
a distal end;
a proximal end;
a pressing segment being formed from the proximal end of the brace segment, being mounted pivotally on the resilient sleeve, corresponding to and extending over the ankle and having a middle;
a support cavity being formed inwardly by shaping the middle of the pressing segment convex; and
a connecting segment being formed from the distal end of the brace segment, being perpendicular to the pressing segment, being mounted around the resilient sleeve and comprising an outer surface.

7. The ankle guard as claimed in claim 6, wherein the connecting segment further has a mounting strip being attached securely to the outer surface of the connecting segment.

8. The brace segment for an ankle guard as claimed in claim 7, wherein
the resilient sleeve further has two mounting holes being formed respectively through the halves of the resilient sleeve near the bottom surface of the resilient sleeve and aligning with each other; and
each brace segments further has
a mounting hole being formed through the pressing segment near the proximal end of the brace segment and aligning to the mounting holes of the resilient sleeve; and
a fastener extending through the mounting holes of the brace segment and the resilient sleeve.

9. The brace segment for an ankle guard as claimed in claim 8, wherein 8 the resilient sleeve farther has two openings being formed respectively in the first and second ends of the resilient sleeve and communicating with each other.

10. The brace segment for an ankle guard as claimed in claim 9, wherein the mounting strip is a hook and loop fastener.

11. The brace segment for an ankle guard as claimed in claim 10, wherein the fastener is a rivet.

12. The brace segment for an ankle guard as claimed in claim 11, wherein the fastener is plastic.
